(19) United States
(12) Patent Application Publication
(10) Pub. No.: US 2004/0267071 A1
(43) Pub. Date: Dec. 30, 2004

(54) Process for the oligomerization of olefins in Fischer-Tropsch derived feeds

(75) Inventors: Thomas V. Harris, Benicia, CA (US); Michael Cheng, Berkeley, CA (US); Guan-Dao Lei, Walnut Creek, CA (US)

Correspondence Address: CHEVRON TEXACO CORPORATION, P.O. BOX 6006, SAN RAMON, CA, 94583-0806, US

(73) Assignee: Chevron U.S.A. Inc.

(21) Appl. No.: 10/611,776

(22) Filed: Jun. 30, 2003

(51) Int. Cl. C07C 2/00; C07C 2/02

(52) U.S. Cl. 585/329

(57) ABSTRACT

A process for oligomerizing the olefins present in a Fischer-Tropsch derived condensate containing a mixture of olefins and oxygenates which comprises (a) reducing significantly the oxygenates present in the Fischer-Tropsch condensate; (b) contacting the Fischer-Tropsch derived condensate having significantly reduced oxygenates with an ionic liquid catalyst in an oligomerization zone under oligomerization reaction conditions; and (c) recovering from the oligomerization zone a Fischer-Tropsch derived product having molecules characterized by a higher average molecular weight and increased branching as compared to the Fischer-Tropsch derived condensate.

### FIELD OF THE INVENTION

This invention relates to the oligomerization of olefins present in Fischer-Tropsch derived feeds by use of an ionic liquid oligomerization catalyst.

### BACKGOUND OF THE INVENTION

The economics of a Fischer-Tropsch complex has in the past only been desirable in isolated areas where it is impractical to bring the natural gas to market; however, a Fischer-Tropsch complex can benefit if the production of high-value products in the product slate, such as lubricating base oil and high quality diesel, can be increased. Fortunately, the market for lubricating base oils of high paraffinicity is continuing to grow due to the high viscosity index, oxidation stability, and low volatility relative to viscosity of these molecules. The products produced from the Fischer-Tropsch process contain a high proportion of wax which makes them ideal candidates for processing into lube base stocks. Accordingly, the hydrocarbon products recovered from the Fischer-Tropsch process have been proposed as feedstocks for preparing high quality lube base oils.

If desired, high quality diesel products also may be prepared from the syncrude recovered from the Fischer-Tropsch process. Fischer-Tropsch derived diesel typically has very low sulfur and aromatics content and an excellent cetane number. In addition, the process of the present invention makes it possible to produce diesel having low pour and cloud points which enhance the quality of the product. These qualities make Fischer-Tropsch derived diesel an excellent blending stock for upgrading lower quality petroleum-derived diesel.

Accordingly, it is desirable to be able to maximize the yields of such higher value hydrocarbon products which boil within the range of lubricating base oils and diesel. At the same time, it is desirable to minimize the yields of lower value products such as naphtha and C₄ minus products. Unfortunately, most Fischer-Tropsch processes produce lower molecular weight olefinic products within the C₃ to C₈ range. The present invention makes it possible to increase the yield of higher boiling products and also increase the amount of branching in the molecules.

All syncrude Fischer-Tropsch products as they are initially recovered from the Fischer-Tropsch reactor contain varying amounts of olefins depending upon the type of Fischer-Tropsch operation employed. In addition, the crude Fischer-Tropsch product also contains a certain amount of oxygenated hydrocarbons, especially alcohols, which may be readily converted to olefins by a dehydration step. These olefins may be oligomerized to yield hydrocarbons having a higher molecular weight than the original feed. Oligomerization also introduces desirable branching into the hydrocarbon molecule which lowers the pour point of the diesel and lubricating base oil products, thereby improving the cold flow properties of the product. See, for example, U.S. Pat. No. 4,417,088. For those Fischer-Tropsch products intended as feed for a hydrocracking operation, a further advantage is that the branching renders the molecule easier to crack. Most of the oxygenates from the Fischer-Tropsch operation will be included in the condensate fraction recovered from the unit. As used in this disclosure, the term “Fischer-Tropsch condensate” refers generally to the C₅ plus fraction which has a lower boiling point than the Fischer-Tropsch wax fraction. That is to say, the condensate represents that fraction which is normally liquid at ambient temperature. In contrast, “Fischer-Tropsch wax” refers to the high boiling fraction from the Fischer-Tropsch derived syncrude and is most often a solid at room temperature.

One method for introducing branching into Fischer-Tropsch-derived products is to oligomerize the olefins which are present in the condensate recovered from the Fischer-Tropsch reactor. The oligomerization of olefins introduces branching into the carbon backbone. As already noted, branching results in desirable lubricating properties. U.S. Pat. No. 4,417,088 describes a process for oligomerizing olefins to produce molecules having desirable branching. In addition, oligomerization increases the yield of higher boiling products, such as lubricating base oils and diesel, and lowers the yield of lower boiling products, such as LPG and naphtha, from the Fischer-Tropsch operation. Recently, the use of ionic liquid catalysts has been proposed for use in the oligomerization of olefins. See, for example, U.S. Pat. Nos. 5,304,615 and 5,463,158. See also European Patent Application No. EP 0791643 A1. U.S. Pat. No. 6,395,948 teaches that the oligomerization of alphaolefins using an ionic liquid catalyst must be conducted in the absence of an organic diluent if a polyalphaolefin having a high viscosity is desired.

Applicants have found that the presence of oxygenates interferes with the oligomerization of olefins when an ionic liquid catalyst is used. Therefore, Applicants have found that it is necessary to remove the oxygenates from the feed prior to the oligomerization step, such as by use of an adsorbent. X-type zeolites, especially 13X zeolite, have been found to be particularly useful in carrying out the present invention. U.S. Pat. No. 2,882,244 discloses the use of X zeolites as adsorbents. The use of 13X zeolite as an adsorbent is taught U.S. Pat. No. 4,481,018 to Coe et al.

As used in this disclosure, the words “comprises” or “comprising” is intended as an open-ended transition meaning the inclusion of the named elements, but not necessarily excluding other unnamed elements. The phrases “consists essentially of” or “consisting essentially of” are intended to mean the exclusion of other elements of any essential significance to the composition. The phrases “consisting of” or “consists of” are intended as transitions meaning the exclusion of all but the recited elements with the exception of only minor traces of impurities.

### BRIEF DESCRIPTION OF THE INVENTION

In its broadest aspect, the present invention is directed to a process for oligomerizing the olefins present in a Fischer-Tropsch derived condensate containing a mixture of olefins and oxygenates which comprises (a) reducing significantly the oxygenates present in the Fischer-Tropsch condensate; (b) contacting the Fischer-Tropsch derived condensate having significantly reduced oxygenates with an ionic liquid catalyst in an oligomerization zone under oligomerization reaction conditions; and (c) recovering from the oligomerization zone a Fischer-Tropsch derived product having molecules characterized by a higher average molecular weight and increased branching as compared to the Fischer-Tropsch derived condensate.

The present invention is also directed to a process for preparing a Fischer-Tropsch derived product by the oligomerization of the olefins in a Fischer-Tropsch derived concentrate which contains olefins and oxygenates which comprises (a) dehydrating the Fischer-Tropsch derived concentrate in a dehydration zone under dehydration conditions and recovering a dehydrated Fischer-Tropsch derived condensate from the dehydration zone; (b) contacting the dehydrated Fischer-Tropsch derived condensate with a molecular sieve capable of adsorbing the oxygenates remaining in the dehydrated Fischer-Tropsch derived condensate and recovering a Fischer-Tropsch derived condensate intermediate containing significantly reduced oxygenates; (c) contacting the Fischer-Tropsch derived condensate intermediate in an oligomerization zone with an effective oligomerizing amount of an acidic ionic liquid oligomerization catalyst while maintaining said Fischer-Tropsch derived condensate intermediate and said oligomerization catalyst under preselected oligomerization conditions for a sufficient time to oligomerize the olefins present; and (d) recovering from the oligomerization zone a Fischer-Tropsch derived product having molecules characterized by a higher average molecular weight and increased branching as compared to the Fischer-Tropsch derived condensate.

It has been found that oxygenates present in Fischer-Tropsch derived feeds interfere with the ability of an ionic liquid oligomerization catalyst to promote the oligomerization of the olefins present in the condensate. Surprisingly, this interference occurs even when the Fischer-Tropsch feed is first subjected to a dehydration step which converts substantially all of the alcohols present into olefins. It has been discovered that even low levels of other oxygenates, such as ketones and carboxylic acids, which remain in the condensate after the dehydration step will deactivate the ionic liquid catalyst. Therefore, it is essential when an ionic liquid catalyst is employed to oligomerize the olefins in the condensate fraction to significantly the remaining oxygenates present. Preferably, substantially all of the remaining oxygenates are removed prior to oligomerization.

Any of a number of methods may be used for removing the oxygenates from Fischer-Tropsch derived feeds. For example, the addition of sodium metal to the condensate may be employed to reduce the oxygenates. A more commercially practical way of removing oxygenates is by the use of an adsorbent, such as, for example, a molecular sieve having a low silica to alumina ratio. Large pore molecular sieves having low silica to alumina ratio, particularly those molecular sieves characterized as having an FAU type of framework, may be suitable for use as an adsorbent for oxygenates. Preferred FAU molecular sieves are X zeolites, with 13X zeolite being particularly preferred.

Following removal of the oxygenates, the olefins in the condensate are oligomerized using an effective oligomerizing amount of a Lewis acid ionic liquid catalyst.

Following oligomerization, it is usually desirable to saturate the remaining double bonds in the hydrocarbon molecules of the Fischer-Tropsch derived products. This operation, referred to herein as hydrofinishing, improves the UV and oxygen stability of the products.

### DETAILED DESCRIPTION OF THE INVENTION

As noted above, the oligomerization of the olefins normally present in the condensate recovered from a Fischer-Tropsch operation increases the production of higher value products, such as lubricating base oils and diesel, and also introduces desirable branching into the molecules which helps to improve the cold flow properties of the products. The use of an ionic liquid catalyst for the oligomerization of the olefins in the condensate has certain advantages over more conventional catalysts, in that there is excellent mixing of the reactants with the catalyst resulting in short residence times and high yields, the oligomerization reactions takes place at relatively low temperatures, and the products are readily separated from the catalyst. However, it has been found that the oxygenates normally present in the Fischer-Tropsch condensate deactivate the catalyst unless they are removed prior to the oligomerization operation. Initially, the oxygenates were not believed to present a major problem, since the condensate recovered from the Fischer-Tropsch operation is usually subjected to a dehydration step prior to the oligomerization step in order to convert substantially all of the alcohols present into olefins. Since most of the oxygenates present in the condensate are represented by alcohols, it was believed that further processing of the condensate was unnecessary prior to oligomertization. However, it was found that other oxygenates were present and even at very low levels deactivated the catalyst. These oxygenates were found to either be passing through the dehydration step unchanged or were being produced in the dehydration step from the alcohols present. Aside from the alcohols, the most important contaminants were found to be ketones and carboxylic acids, with aldehydes, and anhydrides perhaps also causing problems. Therefore, it was found to be essential to include an additional step between the dehydration operation and the oligomerization operation to remove the remaining oxygenates when an ionic liquid catalyst is being utilized.

### Fischer-Tropsch Synthesis

During Fischer-Tropsch synthesis, liquid and gaseous hydrocarbons are formed by contacting a synthesis gas (syngas) comprising a mixture of hydrogen and carbon monoxide with a Fischer-Tropsch catalyst under suitable temperature and pressure reactive conditions. The Fischer-Tropsch reaction is typically conducted at temperatures of from about 300 degrees to about 700 degrees F. (about 150 degrees to about 370 degrees C.), preferably from about 400 degrees to about 550 degrees F. (about 205 degrees to about 290 degrees C.); pressures of from about 10 to about 600 psia (0.7 to 41 bars), preferably 30 to 300 psia (2 to 21 bars); and catalyst space velocities of from about 100 to about 10,000 cc/g/hr., preferably 300 to 3,000 cc/g/hr.

The products from the Fischer-Tropsch synthesis may range from C₁ to C₂₀₀ plus hydrocarbons with a majority in the C₅-C₁₀₀ plus range. The reaction can be conducted in a variety of reactor types, such as, for example, fixed bed reactors containing one or more catalyst beds, slurry reactors, fluidized bed reactors, or a combination of different types of reactors. Such reaction processes and reactors are well known and documented in the literature. The slurry Fischer-Tropsch process, which is preferred in the practice of the invention, utilizes superior heat (and mass) transfer characteristics for the strongly exothermic synthesis reaction and is able to produce relatively high molecular weight paraffinic hydrocarbons when using a cobalt catalyst. In the slurry process, a syngas comprising a mixture of hydrogen and carbon monoxide is bubbled up as a third phase through a slurry which comprises a particulate Fischer-Tropsch type hydrocarbon synthesis catalyst dispersed and suspended in a slurry liquid comprising hydrocarbon products of the synthesis reaction which are liquid under the reaction conditions. The mole ratio of the hydrogen to the carbon monoxide may broadly range from about 0.5 to about 4, but is more typically within the range of from about 0.7 to about 2.75 and preferably from about 0.7 to about 2.5. A particularly preferred Fischer-Tropsch process is taught in European Patent Application No. EP 0609079, also completely incorporated herein by reference for all purposes.

Suitable Fischer-Tropsch catalysts comprise one or more Group VIII catalytic metals such as Fe, Ni, Co, Ru and Re, with cobalt being preferred. Additionally, a suitable catalyst may contain a promoter. Thus, a preferred Fischer-Tropsch catalyst comprises effective amounts of cobalt and one or more of Re, Ru, Pt, Fe, Ni, Th, Zr, Hf, U, Mg and La on a suitable inorganic support material, preferably one which comprises one or more refractory metal oxides. In general, the amount of cobalt present in the catalyst is between about 1 and about 50 weight percent of the total catalyst composition. The catalysts can also contain basic oxide promoters such as ThO₂, La₂O₃, MgO, and TiO₂, promoters such as ZrO₂, noble metals (Pt, Pd, Ru, Rh, Os, Ir), coinage metals (Cu, Ag, Au), and other transition metals such as Fe, Mn, Ni, and Re. Suitable support materials include alumina, silica, magnesia and titania or mixtures thereof. Preferred supports for cobalt containing catalysts comprise alumina or titania. Useful catalysts and their preparation are known and illustrated in U.S. Pat. No. 4,568,663, which is intended to be illustrative but non-limiting relative to catalyst selection.

The products as they are recovered from the Fischer-Tropsch operation usually may be divided into three fractions, a gaseous fraction consisting of very light products, a condensate fraction generally boiling in the range of naphtha and diesel, and a high boiling Fischer-Tropsch wax fraction which is normally solid at ambient temperatures.

### Dehydration

Although the dehydration step is not essential to the present invention, it is advantageous to enrich the condensate with olefins in order to increase the production of higher molecular products. In order to enrich the condensate with olefins, the alcohols may be dehydrated to convert them into olefins prior to the oligomerization step. In general, the dehydration of alcohols may be accomplished by processing the feedstock over a catalyst, such as gamma alumina. Dehydration of alcohols to olefins is discussed in Chapter 5, “Dehydration” in Catalytic Processes and Proven Catalysts by Charles L. Thomas, Academic Press, 1970.

### Removal of Oxygenates

The condensate recovered from the Fischer-Tropsch operation will contain varying amounts of oxygenates. The majority of the oxygenates present in the condensate are in the form of alcohols; however, lesser amounts of ketones, aldehydes, carboxylic acids, and anhydrides may also be present. As already noted above, the presence of even small amounts of oxygenates in the feed to the oligomerization operation will result in the deactivation of the ionic liquid catalyst. Although substantially all of the alcohols present in the condensate will be converted to olefins in the dehydration step, it has been found that dehydration is insufficient to remove all of the oxygenates and that sufficient oxygenates will be present in the effluent from the dehydration step to damage the ionic liquid catalyst. Most of these residual oxygenates are believed to be ketones and carboxylic acids. The oxygenate species remaining after dehydration are believed to vary depending on the source of the condensate. For condensate prepared using an iron-based catalyst, the oxygenate species remaining are primarily ketones. For condensate collected from a Fischer-Tropsch operation using a cobalt-based catalyst, the oxygenates appear to be primarily carboxylic acids. It is unclear whether these residual oxygenates result from the failure of the dehydration step to remove them or if some are actually being produced from the alcohols during the dehydration reaction.

The removal of the oxygenates may be accomplished in various ways, some of which have been previously described in the literature. For example, the oxygenates may be removed by contacting the condensate with sodium metal. While effective, this method is not practical on a commercial scale. A commercially acceptable method for removing the oxygenates involves passing the condensate through an adsorption bed containing an adsorbent capable of adsorbing the oxygenates. A satisfactory adsorbent may include a molecular sieve having low silica to alumina ratio. Large pore molecular sieves having a low silica to alumina ratio, particularly those molecular sieves characterized as having an FAU type of framework, are generally suitable for use as an adsorbent for oxygenates. Preferred FAU molecular sieves are X zeolites, with 13X zeolite being particularly preferred. As used herein, the term “FAU molecular sieve” refers to the IZA Structure Commission standard which includes both X and Y zeolites.

The synthesis of X-type zeolites is described in U.S. Pat. Nos. 2,882,244; 3,685,963; 5,370,879; 3,789,107 and 4,007,253 which are hereby incorporated herein by reference in their entirety. 13X Zeolite are a faujasite (FAU) type X zeolite. It has a low silica/alumina ratio and is comprised of silicon, aluminum and oxygen. The oxygen ring provides a cavity opening of 7.4 angstroms, but can adsorb molecules up to 10 angstroms. 13X zeolite have a Chemical Abstracts (CAS) number of [63231-69-6]. 13X zeolite are commercially available from several sources, including Aldrich Chemical Company and the Davison Division of W. R. Grace.

In practicing the present invention, the amount of the oxygenates are significantly reduced in the Fischer-Tropsch derived condensate prior to the oligomerization step. As used herein, “significantly reduced” means that the elemental oxygen remaining in the Fischer-Tropsch derived condensate is about 1500 ppmw or less. Preferably, substantially all of oxygenates are removed prior to oligomerization. Generally, the Fischer-Tropsch condensate should contain less than about 200 ppm elemental oxygen, even more preferably less than 100 ppm elemental oxygen prior to the oligomerization step.

### Oligomerization

The use of an ionic liquid catalyst for the oligomerization of the olefins in the present invention has certain advantages over more conventional catalysts, in that there is excellent mixing of the reactants with the catalyst resulting in short residence times and high yields, the oligomerization reaction takes place at relatively low temperatures, and the products are readily separated from the catalyst. As noted above, it is essential that the oxygenates present in the feed to the ionic liquid oligomerization operation be reduced to the lowest practical level. The condensate following removal of the oxygenates will consist essentially of an olefin enriched hydrocarbon feed composed mostly of molecules containing between about 5 and about 19 carbon atoms, i.e., that fraction which is normally liquid at ambient temperature. Stated differently, the condensate will comprise primarily saturated and unsaturated hydrocarbons boiling within the range of naphtha and diesel. The Fischer-Tropsch condensate containing the reduced amount of oxygenates may be added to the catalytic mixture or the catalyst may be added to the condensate feed. In either case, the feed and the product formed during oligomerization will form a separate phase from the ionic liquid which allows the product to be readily separated from the ionic liquid catalyst. In order to facilitate mixing of the ionic liquid catalyst and the feed, it is desirable to either stir the oligomerization mixture or bubble the condensate feed through the ionic liquid catalyst. Following completion of the oligomerization reaction, the mixing should be halted, and the product and residual feed should be allowed to form a distinct layer apart from the catalyst phase.

The ionic liquid oligomerization catalyst used in this invention will be a Lewis acid catalyst and usually will comprise at least two components which form a complex. In most instances, the catalyst will be a binary catalyst, i.e., it will consist of only two components. The first component of the catalyst will usually comprise a Lewis acid selected from the group consisting of aluminum halide, alkyl aluminum halide, gallium halide, and alkyl gallium halide. Preferred for the first component is an aluminum halide or alkyl aluminum halide. Aluminum trichloride is particularly preferred for preparing the oligomerization catalyst used in practicing the present invention. The presence of the first component should give the ionic liquid a Lewis (or Franklin) acidic character.

The second component making up the catalyst is usually a quaternary ammonium or quaternary phosphonium compound, such as, for example, a salt selected from one or more of hydrocarbyl substituted ammonium halides, hydrocarbyl substituted imidizolium halide, hydrocarbyl substituted pyridinium halide, alkylene substituted pyridinium dihalide, hydrocarbyl substituted phosphonium halide. Preferred for use as the second component are those quaternary ammonium halides containing one or more alkyl moieties having from 1 to about 9 carbon atoms, such as, for example, trimethylamine hydrochloride, methyl-tributyl ammonium chloride, or alkyl substituted imidazolium halides, such as, for example, 1-ethyl-3-methyl-imidazolium chloride.

The mole ratio of the two components will usually fall within the range of from about 1:1 to about 5:1 of said first component to said second component, and more preferably the mole ratio will be in the range of from about 1:1 to about 2:1. The use of a binary catalyst composition consisting essentially of methyl-tributyl ammonium chloride and aluminum trichloride is particularly advantageous for carrying out the process of the present invention due to the ease of preparation, the ready commercial availability of the components, and the relatively low cost.

The amount of catalyst present to promote the oligomerization of the olefins should be not less than an effective oligomerizing amount, that is to say, the minimum amount of the catalyst necessary to olgomerize the olefins to the desired product. This may vary to some degree depending on the composition of the catalyst, the ratio of the two components of the catalyst to one another, the feed, the oligomerzation conditions chosen, and the like. However, a determination of the effective catalytic amount should be well within the ability of one skilled in the art with no more than routine testing necessary to establish the amount needed to carry out the invention. As noted above, make-up catalyst added to the oligomerization zone may be necessary to replace catalyst that is deactivated by contaminants in the feed, mostly residual oxygenates present in the wax fraction. The amount of make-up catalyst necessary will depend on the amount of contaminants present. Preferably, the amount of contaminants will be low and the degree of deactivation of the catalyst also will be low.

The oligomerization reaction takes place over a wide temperature range between the melting point of the catalyst and its decomposition temperature, preferably between about 120 degrees F. and about 212 degrees F. (about 50 degrees C. and about 100 degrees C.).

Following completion of the oligomerization reaction, the organic layer containing the Fischer-Tropsch derived oligomerization product is separated from the ionic liquid phase. Preferably, the oligomerization product will have an average molecular weight at least 10 percent higher than the initial olefin-enriched Fischer-Tropsch feedstock, more preferably at least 20 percent higher. The acidic ionic liquid catalyst that remains after recovery of the organic phase is preferably recycled to the oligomerization zone.

### Hydrofinishing

Hydrofinishing operations are intended to improve the UV stability and color of the Fischer-Tropsch derived products recovered from the oligomerization zone. It is believed this is accomplished by saturating the double bonds present in the hydrocarbon molecule. A general description of the hydrofinishing process may be found in U.S. Pat. Nos. 3,852,207 and 4,673,487. As used in this disclosure, the term “UV stability” refers to the stability of the lubricating base oil or other products when exposed to ultraviolet light and oxygen. Instability is indicated when a visible precipitate forms or darker color develops upon exposure to ultraviolet light and air which results in a cloudiness or floc in the product. Lubricating base oils and diesel products prepared by the process of the present invention will require UV stabilization before they are suitable for use in the manufacture of commercial lubricating oils and marketable diesel.

In the present invention, the total pressure in the hydrofinishing zone will be above 500 psig, preferably above 1000 psig, and most preferably will be above 1500 psig. The maximum total pressure is not critical to the process, but due to equipment limitations the total pressure will not exceed 3000 psig and usually will not exceed about 2500 psig. Temperature ranges in the hydrofinishing zone are usually in the range of from about 300 degrees F. (150 degrees C.) to about 700 degrees F. (370 degrees C.), with temperatures of from about 400 degrees F. (205 degrees C.) to about 500 degrees F. (260 degrees C.) being preferred. The LHSV is usually within the range of from about 0.2 to about 2.0, preferably 0.2 to 1.5, and most preferably from about 0.7 to 1.0. Hydrogen is usually supplied to the hydrofinishing zone at a rate of from about 1000 to about 10,000 SCF per barrel of feed. Typically, the hydrogen is fed at a rate of about 3000 SCF per barrel of feed.

Suitable hydrofinishing catalysts typically contain a Group VIII noble metal component together with an oxide support. Metals or compounds of the following metals are contemplated as useful in hydrofinishing catalysts include ruthenium, rhodium, iridium, palladium, platinum, and osmium. Preferably, the metal or metals will be platinum, palladium or mixtures of platinum and palladium. The refractory oxide support usually consists of silica-alumina, silica-alumina-zirconia, and the like. Typical hydrofinishing catalysts are disclosed in U.S. Pat. Nos. 3,852,207; 4,157,294 and 4,673,487.

What is claimed is:

1. A process for oligomerizing the olefins present in a Fischer-Tropsch derived condensate containing a mixture of olefins and oxygenates which comprises:
(a) reducing significantly the oxygenates present in the Fischer-Tropsch condensate;
(b) contacting the Fischer-Tropsch derived condensate having significantly reduced oxygenates with an ionic liquid catalyst in an oligomerization zone under oligomerization reaction conditions; and
(c) recovering from the oligomerization zone a Fischer-Tropsch derived product having molecules characterized by a higher average molecular weight and increased branching as compared to the Fischer-Tropsch derived condensate.

2. The process of claim 1 wherein substantially all of the oxygenates present in the Fischer-Tropsch derived condensate are removed.

3. The process of claim 1 wherein the Fischer-Tropsch derived condensate contains not more than about 200 ppmw elemental oxygen.

4. The process of claim 3 wherein the Fischer-Tropsch derived condensate contains not more than about 100 ppmw elemental oxygen.

5. The process of claim 1 wherein the oxygenates are removed by contacting the Fischer-Tropsch derived condensate with an adsorbent which is effective for removing the oxygenates.

6. The process of claim 5 wherein the adsorbent is a molecular sieve having low silica to alumina ratio.

7. The process of claim 6 wherein the molecular sieve is a large pore zeolite.

8. The process of claim 6 wherein the molecular sieve has an FAU type framework.

9. The process of claim 7 wherein the molecular sieve is an X zeolite.

10. The process of claim 7 wherein the molecular sieve is a 13X molecular sieve.

11. A process for preparing a Fischer-Tropsch derived product by the oligomerization of the olefins in a Fischer-Tropsch derived concentrate which contains olefins and oxygenates which comprises:
(a) dehydrating the Fischer-Tropsch derived concentrate in a dehydration zone under dehydration conditions and recovering a dehydrated Fischer-Tropsch derived condensate from the dehydration zone;
(b) contacting the dehydrated Fischer-Tropsch derived condensate with a molecular sieve capable of adsorbing the oxygenates remaining in the dehydrated Fischer-Tropsch derived condensate and recovering a Fischer-Tropsch derived condensate intermediate containing significantly reduced oxygenates;
(c) contacting the Fischer-Tropsch derived condensate intermediate in an oligomerization zone with an effective oligomerizing amount of a Lewis acid ionic liquid oligomerization catalyst while maintaining said Fischer-Tropsch derived condensate intermediate and said oligomerization catalyst under preselected oligomerization conditions for a sufficient time to oligomerize the olefins present; and
(d) recovering from the oligomerization zone a Fischer-Tropsch derived product having molecules characterized by a higher average molecular weight and increased branching as compared to the Fischer-Tropsch derived condensate.

12. The process of claim 11 wherein substantially all of the oxygenates present in the dehydrated Fischer-Tropsch derived condensate are removed.

13. The process of claim 11 wherein the dehydrated Fischer-Tropsch derived condensate contains not more than about 200 ppmw elemental oxygen.

14. The process of claim 13 wherein the dehydrated Fischer-Tropsch derived condensate contains not more than about 100 ppmw elemental oxygen.

15. The process of claim 11 wherein the adsorbent of step (b) is a molecular sieve having low silica to alumina ratio.

16. The process of claim 15 wherein the molecular sieve of step (b) has an FAU type framework.

17. The process of claim 16 wherein the molecular sieve is an X zeolite.

18. The process of claim 16 wherein the molecular sieve of step (b) is a 3X molecular sieve.

19. The process of claim 11 wherein the Lewis acid ionic oligomerization catalyst comprises a first component and a second component, said first component comprising a compound selected from the group consisting of aluminum halide, alkyl aluminum halide, gallium halide, and alkyl gallium halide, and said second component is quaternary ammonium or quaternary phosporium salt.

20. The process of claim 19 wherein said first component is aluminum halide or alkyl aluminum halide.

21. The process of claim 20 wherein said first component is aluminum trichloride.

22. The process of claim 19 wherein said second component is selected from one or more of hydrocarbyl substituted ammonium halide, hydrocarbyl substituted imidazolium halide, hydrocarbyl substituted pyridinium halide, alkylene substituted pyridinium dihalide, or hydrocarbyl substituted phosphonium halide.

23. The process of claim 22 wherein the second component is an alkyl substituted quaternary ammonium halide containing one or more alkyl moieties having from 1 to about 9 carbon atoms.

24. The process of claim 23 wherein the second component comprises at least trimethylamine hydrochloride.

25. The process of claim 22 wherein the second component is an alkyl substituted imidazolium halide.

26. The process of claim 25 wherein the second component comprises at least 1-ethyl-3-methyl-imidazolium chloride.

27. The process of claim 22 wherein the ratio of first component to the second component of the oligomerization catalyst is within the range of from about 1:1 to about 5:1.

28. The process of claim 19 wherein the ratio of the first component to the second component is within the range of from about 1:1 to about 2:1.

29. The process of claim 1 including the additional step of hydrogenating the unsaturated double bonds present in the Fischer-Tropsch derived product.

30. The process of claim 29 wherein the Fischer-Tropsch derived product includes lubricating base oil.

31. The process of claim 29 wherein the Fischer-Tropsch derived product includes a diesel product.
